(19) United States
(12) Patent Application Publication
(10) Pub. No.: US 2009/0070135 A1
(43) Pub. Date: Mar. 12, 2009

(54) SYSTEM AND METHOD FOR IMPROVING CLAIMS PROCESSING IN THE HEALTHCARE INDUSTRY

(75) Inventors: Pradip Kumar Parida, Mallesh Palya (IN); Trivedi Bodlapati, Bangalore (IN)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 11/852,795

(22) Filed: Sep. 10, 2007

(51) Int. Cl. G06Q 50/00

(52) U.S. Cl. 705/2

(57) ABSTRACT

Certain embodiments of the present invention provide for a system and method for managing clinical documents for claims processing. In an embodiment, the method includes receiving a command to submit an invoice to a payer. The method also includes acquiring a first set of files from an electronic medical records database according to a first set of rules. The first set of rules provide for acquiring a first set of files required for submission to a payer. The method also includes displaying an identifier for the first set of files to a user for approval. The first set of files may be transmitted to the payer. In an embodiment, the method may also include receiving a request for additional information from the payer. A second set of files may be acquired according to a second set of rules. The second set of files may be transmitted to the payer.

### BACKGROUND OF THE INVENTION

The present invention generally relates to a system and method for improving claims processing in the healthcare industry. Particularly, the present invention relates to a system and method for improving the efficiency of electronic document retrieval and submission for claims processing.

In the healthcare industry, a healthcare provider generally submits an invoice for payment to a payer. The healthcare provider may be a doctor's office or hospital, for example. The payer may be an insurance company, for example. In general, the provider may submit an invoice to a payer. The payer may respond by submitting payment to the provider.

It is common, however, for the payer to request additional information from the provider before paying an invoice. For example, the payer may request the provider send the payer certain clinical documents as evidence of the charges to the payer. Currently, a provider that receives such a request from the payer searches for the requested clinical documents and sends the documents to the payer. In an example, the provider may not have submitted the correct documents for the charges on the invoice, and the provider may have to repeat the search for documents. The processing of claims by the provider and payer of the claims may result in multiple exchanges of communication between the payers and providers. The processing of claims may be time consuming and lead to delay and/or rejection of payment resulting in delay in revenue collection and/or loss of revenue to the provider.

Accordingly, a system and method is needed to address the inefficiencies in claims processing. Such a system and method may improve and simplify claims processing as well as reduce the time required for a provider to receive payment.

### SUMMARY OF THE INVENTION

Certain embodiments of the present invention may include a method for managing clinical documents for claims processing. The method includes receiving a command to submit an invoice to a payer. The method also includes acquiring a first set of files from an electronic medical records database according to a first set of rules, wherein the first set of rules provide for acquiring a first set of files required for submission to a payer. The method also includes displaying an identifier for the first set of files to a user for approval, wherein if the user approves of the acquisition of the first set of files, electronically transmitting the first set of files to the payer. The method may also include receiving a request for additional information from the payer. The method may also include acquiring a second set of files from the electronic medical records database according to a second set of rules, wherein the second set of rules provide for acquiring a second set of files that supplement the first set of files for submission to the payer. The method may also include displaying an identifier for the second set of files to a user for approval, wherein if the user approves of the acquisition of the second set of files, electronically transmitting the second set of files to the payer. The request for additional information may include a secondary tag that is used to associate the request with other files. In an embodiment, the second set of files may be associated with the secondary tag of the request. In an embodiment, the second set of files is acquired from the electronic medical records database according to the secondary tag. In an embodiment, the invoice contains a primary tag that is used to associate the invoice with other files. In an embodiment, the first set of files is associated with the primary tag of the invoice. In an embodiment, the first set of files is acquired from the electronic medical records database according to the primary tag.

Certain embodiments of the present invention include a computer readable medium including a set of instructions for execution by a computer. In an embodiment, the set of instructions may include a receiving routine for receiving a command to submit an invoice to a payer for services rendered to a patient. The set of instructions may also include an acquisition routine for acquiring a first set of files from an electronic medical records database according to a first set of rules, wherein the first set of rules provide for acquiring a first set of files required for submission to a payer. The set of instructions may also include a displaying routine for displaying an identifier for the first set of files to a user for approval, wherein if the user approves of the acquisition of the first set of files, electronically transmitting the first set of files to the payer. In an embodiment, the set of instructions may also include a second receipt routine for receiving a request for additional information from the payer. The set of instructions may also include a second acquisition routine for acquiring a second set of files from the electronic medical records database according to a second set of rules, wherein the second set of rules provide for acquiring a second set of files that supplement the first the of files for submission to the payer. The set of instructions may also include a second display routine for displaying an identifier for the second set of files to a user for approval, wherein if the user approves of the acquisition of the second set of files, electronically transmitting the second set of files to the payer. The request for additional information includes a secondary tag that is used to associate the request with the second set of files. The second set of files is acquired from the electronic medical records database according to the secondary tag. The invoice may contain a primary tag that is used to associate the invoice with the first set of files. The first set of files may be acquired from the electronic medical records database according to the primary tag.

Certain embodiments of the present invention include a system for managing clinical documents for claims processing. The system may include a client unit having computer software for receiving a command to submit an invoice to a payer for services rendered to a patient, wherein the invoice includes a primary tag. The client unit acquires a first set of files from an electronic medical records database according to a first set of rules, wherein the first set of rules provide for acquiring a first set of files required for submission to a payer, wherein the first set of files is associated with the primary tag. The system may also include a display unit for displaying an identifier for the first set of files to a user for approval, wherein if the user approves of the acquisition of the first set of files, electronically transmitting the first set of files to the payer. In an embodiment, the client unit has computer software for receiving a request for additional information from the payer, wherein the request includes a secondary tag. The client unit acquires a second set of files from the electronic medical records database according to a second set of rules, wherein the second set of rules provide for acquiring a second set of files that supplement the first the of files for submission to the payer, wherein the second set of files is associated with the secondary tag. The client unit transmits the second set of files to the payer upon user approval.

### BRIEF DESCRIPTION OF THE DRAWINGS

FIG. 1 illustrates a system that may be used for healthcare applications in accordance with an embodiment of the present invention.

FIG. 2 illustrates a method for managing clinical documents for claims processing in accordance with an embodiment of the present invention.

FIG. 3 is a table illustrating the Health Insurance Portability and Accountability Act (“HIPAA”) Transactions and Code Sets that may be used in an embodiment of the present invention.

FIG. 4 illustrates an example of a workflow in accordance with an embodiment of the present invention based on the code sets of FIG. 3.

FIG. 5 illustrates a screen shot that may be representative of a user interface that may be used in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

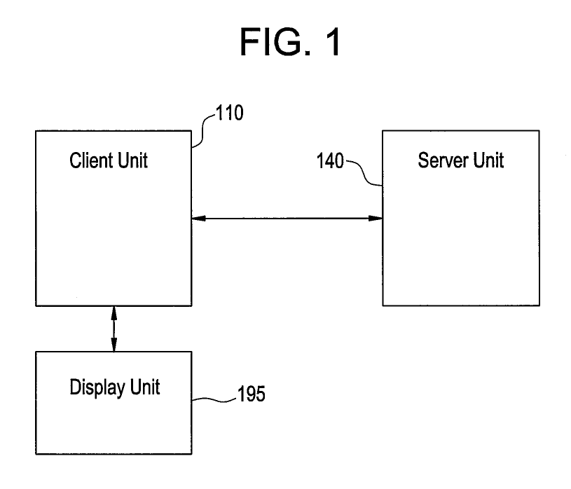

FIG. 1 illustrates a system 100 that may be used for healthcare applications in accordance with an embodiment of the present invention. The system 100 may represent a healthcare provider. The system 100 illustrates a client unit 110. The client unit may be a computer unit, such as a personal computer. The client unit 110 may receive input from a user, for example through a keyboard or computer mouse. Other forms of input may be received, for example voice commands. The client unit may include hardware, firmware, and software. The system 110 may also include a display unit 195. The display unit 195 may be a general computer display unit. The client 110 may be connected to the display unit 195 to display various graphics to a user. The display unit 195 may be a single display unit or multiple display units. Additionally, the display unit 195 may be a two-dimensional display unit or a three-dimensional display unit, for example. Accordingly, any display unit may be used in accordance with the present invention.

The client 110 may also be connected to a server unit 140. The server unit 140 may include hardware, software, firmware, and memory. The client unit 110 may communicate with the server unit 140. The connection between the client unit 110 and the server unit 140 may be wired or wireless. The client unit 110 represents, in general, equipment and software. The actual physical devices of the client unit 110 may be separate units, part of a single unit, a computer system, or part of a computer system. The server unit 140 represents, in general, equipment and software. The actual physical devices of the server unit 140 may be separate units, part of a single unit, a computer system, or part of a computer system. The client unit 110 and server unit 140 may be connected to other devices via an electronic network. The components of the system 100 may be single units, separate units, may be integrated in various forms, and may be implemented in hardware, firmware, and/or in software.

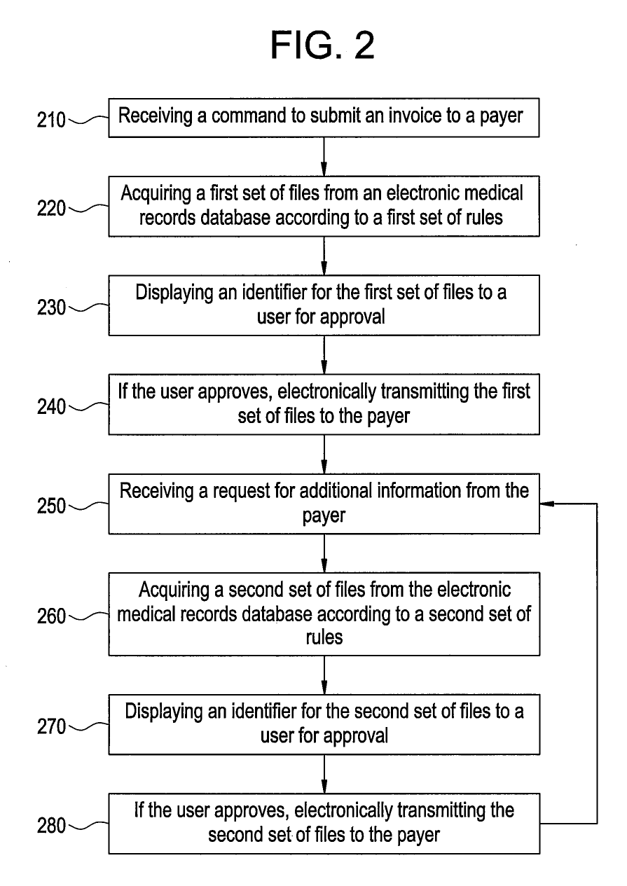

FIG. 2 illustrates a method for managing clinical documents for claims processing in accordance with an embodiment of the present invention. At step 210, a command to submit an invoice to a payer is received. The command to submit an invoice may include information such as the contact information for the payer. In an embodiment, the payer may be an insurance company. For example, the contact information may include an email address. In addition, the invoice may include a claim number, procedure type, patient information, and other information a payer may need to process the claim. In an embodiment, the information in the invoice is coded into a primary tag for the invoice. The primary tag may be used to identify the patient, type of procedure, and the documents associated with the patient and procedure that are relevant to the invoice. The primary tag may be used to associate the invoice with relevant documents and files.

At step 220, a first set of files may be acquired from a server according to a first set of rules. In an embodiment, the server may include an electronic medical records database. In an embodiment, the first set of files includes the documents required by the payer for submission of the claim for the patient. For example, the first set of files may include several files that are evidence that procedure X was performed on patient Y. The first set of rules may be defined based on the procedure being submitted. For example, for procedure X, the first set of rules may dictate that files A, B, and C be acquired. For procedure Z, the first set of rules may dictate that files D, E and F be acquired. The first set of rules is generally defined by the requirements of the payer. In an embodiment, the first set of rules may be different for the same procedure for different payers. For example, if the payer is an insurance company, a patient Y having procedure X and having a payer PI may have a first set of files comprising A, B, and C. A patient Y1 having procedure X and having a payer P2 may have a first set of files comprising A and B. In an embodiment, the primary tag for an invoice is associated with the first set of files. In an example, the primary tag of an invoice is used to acquire the first set of files from an electronic medical records database.

At step 230, once the first set of files is acquired, an identifier for the first set of files may be displayed to a user for approval. In an embodiment, the identifier may be the title of the first set of files. If the user approves of the files that have been acquired, as in step 240, the files are electronically transmitted to the payer for review. Upon receipt of the files, the payer may find the information sufficient to pay the claim and send payment to the healthcare provider. If the payer does not find the information sufficient to pay the claim, the payer may send a request to the healthcare provider for additional information.

At step 250, a request for additional information from the payer may be received. In an embodiment, the request for additional information may include specific requests for certain documents. Alternatively, the request for additional information may include a general request based on the procedure type. At step 260, a second set of files from the electronic medical records database may be acquired according to a second set of rules. In an embodiment, the second set of files may include the files specifically requested by the payer for a particular patient. In such an embodiment, the second set of rules is dictated by the request for additional information. The second set of rules may state to acquire the documents specifically requested. Alternatively, the request for additional information may include a general request based on procedure type. In such an embodiment, the second set of rules dictates a predefined set of documents for a procedure type that provide additional information on the procedure type and evidence for payment. The request for additional documents may include a secondary tag that is used to associate the request with the second set of files. In an embodiment, the second set of files is acquired from an electronic medical records database according to the second set of rules. The second set of rules provide for acquiring a second set of files that supplement the first set of files for submission to the payer. In an embodiment, the second set of files is acquired from the electronic medical records database according to the secondary tag.

At step 270, an identifier for the second set of files may be displayed to a user for approval. In an embodiment, the identifier may include the title of the second set of files for display to the user for approval. If the user approves of the files that have been acquired, as in step 280, the files are electronically transmitted to the payer for review. If the payer finds the second set of files sufficient to pay the claim, the payer may send payment to the healthcare provider. If the payer does not find the information sufficient to pay the claim, the payer may send another request to the healthcare provider for additional information. The procedure of step 250—step 280 may repeat until the payer is satisfied.

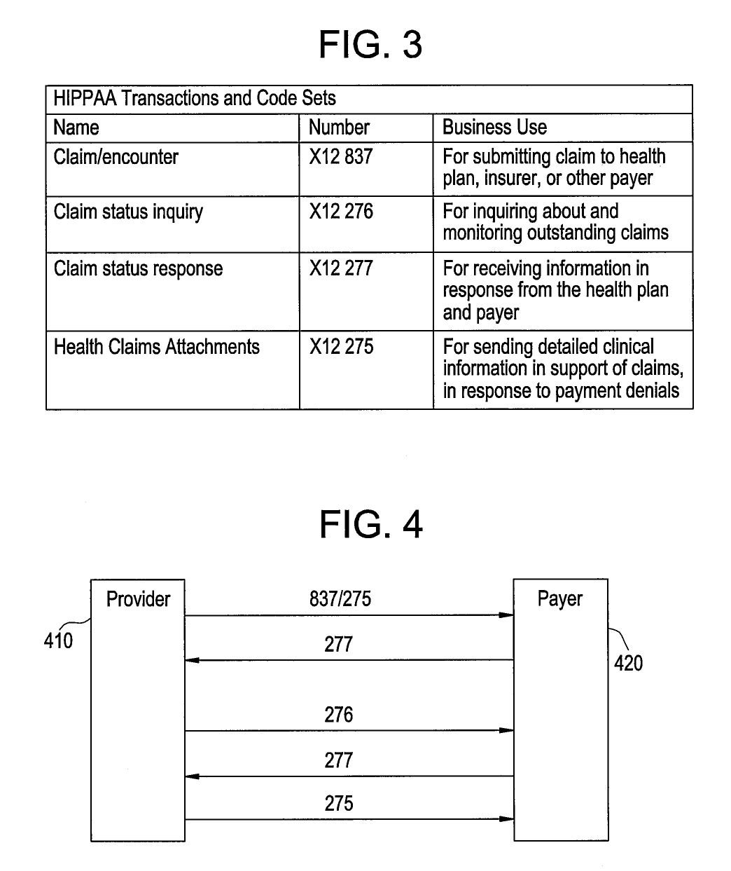

FIGS. 3 and 4 illustrate an example of an embodiment of the present invention in operation. FIG. 3 is a table illustrating the Health Insurance Portability and Accountability Act (“HIPAA”) Transactions and Code Sets that may be used in an embodiment of the present invention. In an embodiment, the first row of FIG. 3 illustrates that the number X12 837 may be used for submitting claims to health plan, insurer, or other payer. The second row of FIG. 3 illustrates that the number X12 276 may be used for inquiring about and monitoring outstanding claims. The third row of FIG. 3 illustrates that the number X12 277 may be used for receiving information in response from the health plan and payer. The fourth row of FIG. 3 illustrates that the number X12 275 may be used for sending detailed clinical information in support of claims, in response to payment denials.

FIG. 4 illustrates an example of a workflow 400 in accordance with an embodiment of the present invention based on the code sets of FIG. 3. Element 410 represents the provider and element 420 represents the payer. In an embodiment, the provider 410 collects the relevant information for a claim. As shown in FIG. 4, the provider 410 may submit to the payer 420 X12 837 and X12 275 requests transmitting the relevant information to the payer 420.

The payer 420 may respond to the X12 837 request with an X12 277 request for additional information for the claim. The X12 277 request is received by the provider 410 and the provider 410 searches for the additional information. In an embodiment, the search may be performed based on the procedure type and a predefined set of rules. In another embodiment, the search may be performed based on documents that are specifically requested.

Once the documents that are responsive to the X12 277 request are acquired, but before the responsive documents are transmitted, the provider 410 may send an X12 276 communication to the payer to inquire about and monitor the outstanding claims. The X12 276 message may be sent to confirm that the additional information is still requested. In response to the X12 276 request, the payer may resend the X12 277 request to confirm the request for additional information. In response, the provider 410 may send an X12 275 message that includes detailed clinical information in support of the claims.

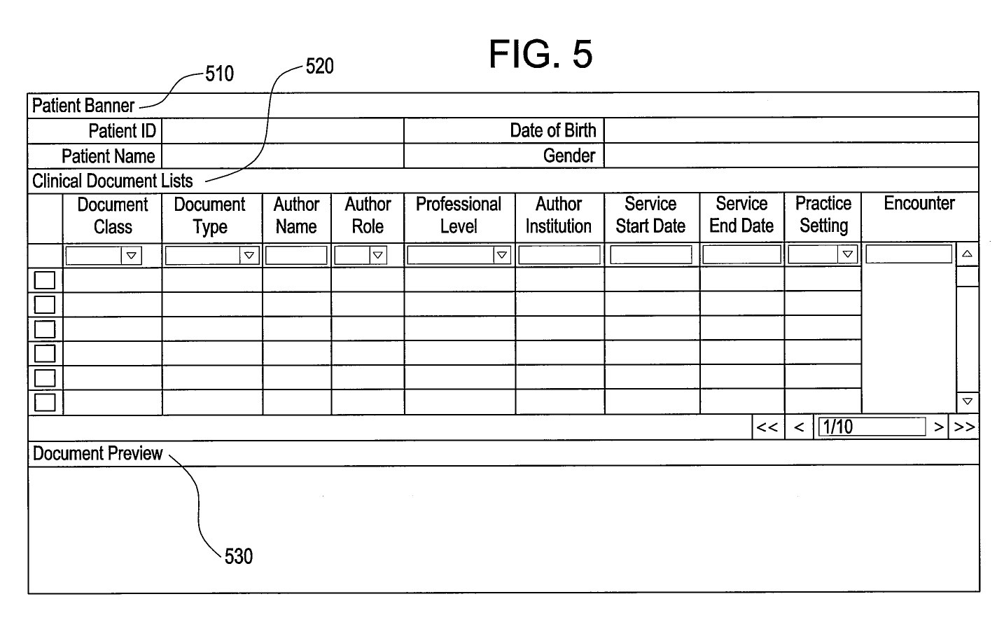

FIG. 5 illustrates a screen shot 500 that may be representative of a user interface that may be used in accordance with an embodiment of the present invention. FIG. 5 illustrates a user interface that may be used as a viewer to review the files that have been acquired prior to submission to the payer. FIG. 5 illustrates a patient banner 510, a document list 520, and a document preview frame 530. The patient banner 510 provides information about the patient. For example, the patient banner 510 may identify the patient ID, patient name, date of birth, and gender. The document list frame 520 may display a list of the acquired documents. The document list frame 520 may also provide the ability to filter the documents based on various attributes. In an embodiment, a user may have the capability to select one or more of the documents in the document list 520 and the selected document display in the document preview frame 530.

The system and method described above may be carried out as part of a computer-readable storage medium including a set of instructions for a computer. The set of instructions may include a receiving routine for receiving a command to submit an invoice to a payer for services rendered to a patient. The set of instructions may also include an acquisition routine for acquiring a first set of files from an electronic medical records database according to a first set of rules. The first set of rules provide for acquiring a first set of files required for submission to a payer. The set of instructions may also include a displaying routine for displaying an identifier for the first set of files to a user for approval. In an embodiment, the identifier may include the title of the first set of files. If the user approves of the acquisition of the first set of files, the first set of files is electronically transmitted to the payer.

The set of instructions also includes a second receipt routine for receiving a request for additional information from the payer. The set of instructions may also include a second acquisition routine for acquiring a second set of files from the electronic medical records database according to a second set of rules. The second set of rules provide for acquiring a second set of files that supplement the first set of files for submission to the payer. The set of instructions may also include a second display routine for displaying an identifier for the second set of files to a user for approval. In an embodiment, the identifier may include the title for the second set of files. If the user approves of the acquisition of the second set of files, the second set of files is electronically transmitted to the payer. The request for additional information may include a secondary tag that is used to associate the request with the second set of files. The second set of files may be acquired from the electronic medical records database according to the secondary tag. The invoice may contain a primary tag that is used to associate the invoice with the first set of files. The first set of files may be acquired from the electronic medical records database according to the primary tag.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for managing clinical documents for claims processing, said method comprising:
receiving a command to submit an invoice to a payer;
acquiring a first set of files from an electronic medical records database according to a first set of rules, wherein said first set of rules provide for acquiring a first set of files required for submission to a payer;
displaying an identifier for said first set of files to a user for approval, wherein if said user approves of the acquisition of said first set of files, electronically transmitting said first set of files to said payer.

2. The method of claim 1, further comprising receiving a request for additional information from said payer.

3. The method of claim 2, further comprising acquiring a second set of files from said electronic medical records database according to a second set of rules, wherein said second set of rules provide for acquiring a second set of files that supplement said first set of files for submission to said payer.

4. The method of claim 3, further comprising displaying an identifier for said second set of files to a user for approval, wherein if said user approves of the acquisition of said second set of files, electronically transmitting said second set of files to said payer.

5. The method of claim 2, wherein said request for additional information includes a secondary tag that is used to associate the request with other files.

6. The method of claim 5, wherein said second set of files is associated with said secondary tag of said request.

7. The method of claim 6, wherein said second set of files is acquire from said electronic medical records database according to said secondary tag.

8. The method of claim 1, wherein said invoice contains a primary tag that is used to associate the invoice with other files.

9. The method of claim 8, wherein said first set of files is associated with said primary tag of said invoice.

10. The method of claim 9, wherein said first set of files is acquired from said electronic medical records database according to said primary tag.

11. A computer readable medium including a set of instructions for execution by a computer, said set of instructions comprising:
a receiving routine for receiving a command to submit an invoice to a payer for services rendered to a patient;
an acquisition routine for acquiring a first set of files from an electronic medical records database according to a first set of rules, wherein said first set of rules provide for acquiring a first set of files required for submission to a payer;
a displaying routine for displaying an identifier for said first set of files to a user for approval, wherein if said user approves of the acquisition of said first set of files, electronically transmitting said first set of files to said payer.

12. The set of instructions of claim 11, further comprising a second receipt routine for receiving a request for additional information from said payer.

13. The set of instructions of claim 12, further comprising a second acquisition routine for acquiring a second set of files from said electronic medical records database according to a second set of rules, wherein said second set of rules provide for acquiring a second set of files that supplement said first said of files for submission to said payer.

14. The set of instructions of claim 13, further comprising a second display routine for displaying an identifier for said second set of files to a user for approval, wherein if said user approves of the acquisition of said second set of files, electronically transmitting said second set of files to said payer.

15. The set of instructions of claim 12, wherein said request for additional information includes a secondary tag that is used to associate the request with said second set of files.

16. The set of instructions of claim 15, wherein said second set of files is acquired from said electronic medical records database according to said secondary tag.

17. The set of instructions of claim 11, wherein said invoice contains a primary tag that is used to associate the invoice with said first set of files.

18. The set of instructions of claim 17, wherein said first set of files is acquired from said electronic medical records database according to said primary tag.

19. A system for managing clinical documents for claims processing, said system comprising:
a client unit having computer software for receiving a command to submit an invoice to a payer for services rendered to a patient, wherein said invoice includes a primary tag;
wherein said client unit acquires a first set of files from an electronic medical records database according to a first set of rules, wherein said first set of rules provide for acquiring a first set of files required for submission to a payer, wherein said first set of files is associated with said primary tag;
a display unit for displaying an identifier for said first set of files to a user for approval, wherein if said user approves of the acquisition of said first set of files, electronically transmitting said first set of files to said payer.

20. The system of claim 19, wherein said client unit has computer software for receiving a request for additional information from said payer, wherein said request includes a secondary tag;
wherein said client unit acquires a second set of files from said electronic medical records database according to a second set of rules, wherein said second set of rules provide for acquiring a second set of files that supplement said first said of files for submission to said payer, wherein said second set of files is associated with said secondary tag;
wherein said client unit transmits the second set of files to said payer upon user approval.
